(19) United States
(12) Patent Application Publication
(10) Pub. No.: US 2013/0296694 A1
(43) Pub. Date: Nov. 7, 2013

(54) SURGICAL INSTRUMENTS FOR OCT ASSISTED PROCEDURES

(71) Applicant: THE CLEVELAND CLINIC FOUNDATION, Cleveland, OH (US)

(72) Inventors: Justis P. Ehlers, Shaker Hts., OH (US); Sunil K. Srivastava, Shaker Hts., OH (US); Yuankai Tao, Cleveland, OH (US)

(73) Assignee: THE CLEVELAND CLINIC FOUNDATION, Cleveland, OH (US)

(21) Appl. No.: 13/887,459

(22) Filed: May 6, 2013

Related U.S. Application Data
(60) Provisional application No. 61/642,990, filed on May 4, 2012.

(51) Int. Cl. A61B 5/06

(52) U.S. Cl. CPC A61B 5/065
USPC 600/424; 600/427

(57) ABSTRACT

Assemblies are provided for use as surgical instruments in optical coherence tomography (OCT) assisted surgical procedures. Each assembly includes a working assembly, formed from a material selected for desirable optical properties or modified to increase the visibility of the material in an OCT scan, and a handle attached to the working assembly.

### RELATED APPLICATION

This application claims priority from U.S. Provisional Patent Application Ser. No. 61/642,990, filed 4 May 2012, the subject matter of which is incorporated hereby incorporated by reference in its entirety.

### TECHNICAL FIELD

The present invention relates generally to the field of medical devices, and more particularly to surgical instruments suitable for optical coherence tomography (OCT) assisted procedures.

### BACKGROUND OF THE INVENTION

Over the years, multiple milestones have revolutionized ophthalmic surgery. X-Y surgical microscope control, wide-angle viewing, and fiber optic illumination are all examples of instrumentation that have been integrated to radically improve pars plana ophthalmic surgery. A major advance in ophthalmic surgery may be the integration of retinal imaging into the operating room. Optical coherence tomography (OCT) has dramatically increased the efficacy of treatment of ophthalmic disease through improvement in diagnosis, understanding of pathophysiology, and monitoring of progression over time. Its ability to provide a high-resolution, cross-sectional, three-dimensional view of the relationships of ophthalmic anatomy during surgery makes intraoperative OCT a logical complement to the ophthalmic surgeon.

### SUMMARY OF THE INVENTION

In accordance with an aspect of the present invention, an assembly is provided for use as a surgical instrument in a surgical procedure guided by an optical coherence tomography (OCT) system having a light source with an associated wavelength within the near infrared range. The assembly includes a working assembly formed from a semi-transparent plastic selected to have an index of refraction between 1.3 and 1.55 at the associated frequency and a scattering coefficient between 2 mm⁻¹ and 5 mm⁻¹ at the associated frequency and a handle attached to the working assembly.

In accordance with another aspect of the present invention, an assembly is provide for use as a surgical instrument in a surgical procedure guided by an optical coherence tomography (OCT) system having a light source with an associated frequency within the near infrared range. The assembly includes a working assembly formed from a semi-transparent plastic doped with a contrast agent selected to improve the visibility of the working assembly in an OCT scan and a handle attached to the working assembly.

In accordance with still another aspect of the present invention, an assembly is provided for use as a surgical instrument in a surgical procedure guided by an optical coherence tomography (OCT) system having a light source with an associated frequency within the near infrared range. The assembly includes a working assembly formed from a semi-transparent plastic, specifically one of glycol modified poly(ethylene terephthalate, polyvinyl chloride, poly(methyl methacrylate), or polyphenylsulfone, and a handle attached to the working assembly.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates an abstract, functional block diagram of a surgical instrument for use in optical coherence tomography (OCT) assisted procedures in accordance with an aspect of the present invention;

FIG. 2 illustrates a first example of a surgical instrument, specifically an ophthalmic pic, in accordance with an aspect of the present invention;

FIG. 3 provides a close-up view of a working assembly associated with the ophthalmic pic;

FIG. 4 illustrates an OCT scan of a region of tissue with the ophthalmic pic of FIGS. 2 and 3 interposed between the OCT scanner and the tissue;

FIG. 5 illustrates a second example of a surgical instrument, specifically ophthalmic forceps, in accordance with an aspect of the present invention;

FIG. 6 provides a close-up view of a working assembly associated with the ophthalmic forceps;

FIG. 7 illustrates an OCT scan of a region of tissue with the ophthalmic forceps of FIGS. 5 and 6 interposed between the OCT scanner and the tissue;

FIG. 8 illustrates a first method for constructing an instrument for use in OCT assisted surgical procedures in accordance with an aspect of the present invention; and

FIG. 9 illustrates a second method for constructing an instrument for use in OCT assisted surgical procedures in accordance with an aspect of the present invention.

### DETAILED DESCRIPTION

Optical Coherence Tomography (OCT) is a non-contact imaging modality that provides high resolution cross-sectional images the eye and its microstructure. This ability to quickly image ophthalmic anatomy as an “optical biopsy” has revolutionized ophthalmology. OCT is the most commonly performed imaging procedure in ophthalmology. The cross-sectional information provided by OCT is a natural complement to the ophthalmic surgeon. Real-time information could improve surgical precision, reduce surgical times, and improve outcomes.

The inventor has found a major limiting factor for the use of OCT in the operating room is the lack of “OCT-friendly” instrumentation. Current materials and instruments are not suitable for OCT imaging due to blockage of light transmission and suboptimal reflectivity profiles limiting visualization of the instrument, underlying tissues, and instrument/tissue interactions. For example, current metallic instruments exhibit absolute shadowing of underlying tissues due to a lack of light transmission. Additionally, the low light scattering properties of metal result in a pinpoint reflection that does not allow for the instrument to be visualized on OCT scanning. Silicone based materials have more optimal OCT reflectivity properties, however, silicone does not provide the material qualities to create the wide-ranging instrument portfolio needed for intraocular surgery (e.g., forceps, scissors, blades).

Instruments in accordance with the present invention provide the next step in surgical instruments allowing for OCT integration into the operating room. Specifically, a working assembly of each instrument can be designed to have optical properties to optimize visualization of underlying tissues while maintaining instrument visualization on the OCT scan. The unique material composition and design of these instruments will maintain the surgical precision for microsurgical manipulations, while providing optimal optical characteristics that allow for intraoperative OCT imaging. The optical features of these materials include a high rate of light transmission to reduce the shadowing of underlying tissue. This allows tissues below the instruments to be visualized on the OCT scans while the instrument hovers above the tissue or approaches the tissue. Simultaneously, the materials can either have light scattering properties that are high enough to allow for visualization of the instrument contours and features on OCT imaging or be doped with an appropriate contrast agent to provide these properties. Where doping is used, a light source separate from the OCT scanner may be used to allow for location of even low concentrations of the doping agent.

Such instruments could be used in potentially all ophthalmic surgical procedures. In particular, intraocular surgeries (e.g., cataract, corneal, ophthalmic) could be impacted tremendously by the availability of intraoperative OCT and the necessary “OCT-friendly” instrumentation. In cataract surgery, OCT assisted corneal incisions could improve wound construction, reducing hypotony and infection rates, as well as confirmation of anatomic location of intraocular lens insertions. In corneal surgery, intraoperative OCT would provide critical information in lamellar surgeries on graft adherence and lamellar architecture. For ophthalmic surgery, OCT-assisted surgery will be critical to guiding membrane peeling in diabetic retinal detachments, macular puckers, and macular holes. Utilizing the instrumentation described herein, real-time scanning could be performed to confirm the correct anatomic localization of instruments (e.g., vessel cannulation, intraocular biopsy, and specific tissue layer) and identify key surgical planes.

The application of these material technologies may be far reaching. OCT technology is now touching numerous fields throughout medicine (e.g., cardiology, dermatology, and gastroenterology). Diagnostic and surgical procedures are using OCT as an adjunct. Application of this invention to new devices within other specialties could broaden the diagnostic and therapeutic utility of OCT across medicine. Accordingly, properly optimized materials could also be utilized to create devices and instruments to be utilized in other areas of medicine which are already using OCT as a diagnostic modality but do not have instrumentation that is compatible with OCT to use it as a real-time adjunct to therapeutic maneuvers.

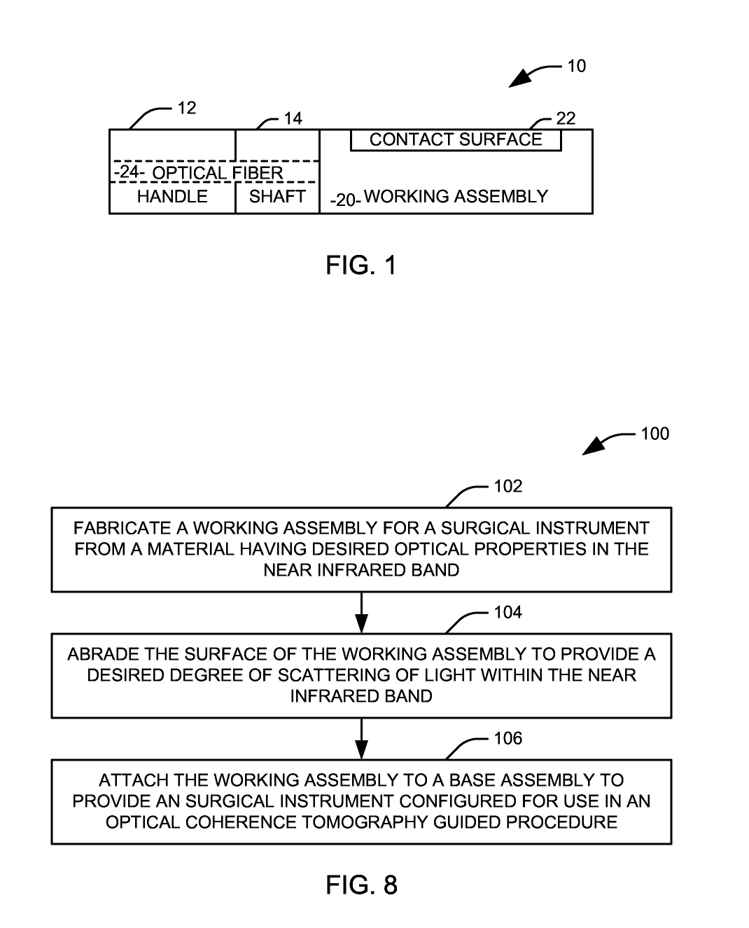

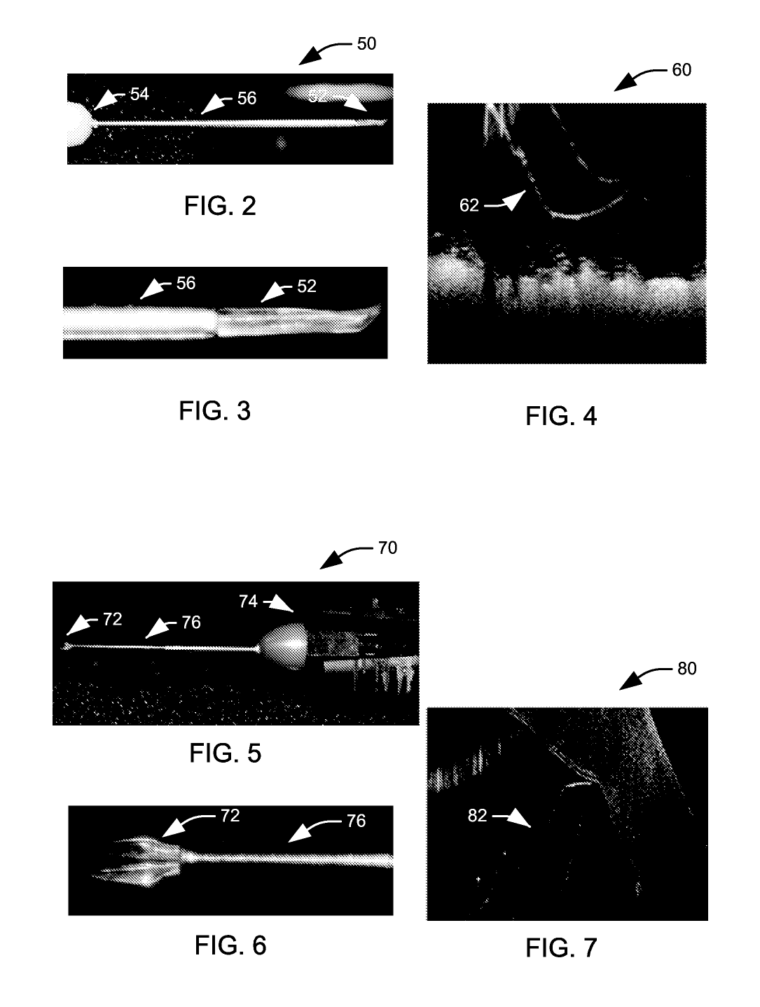

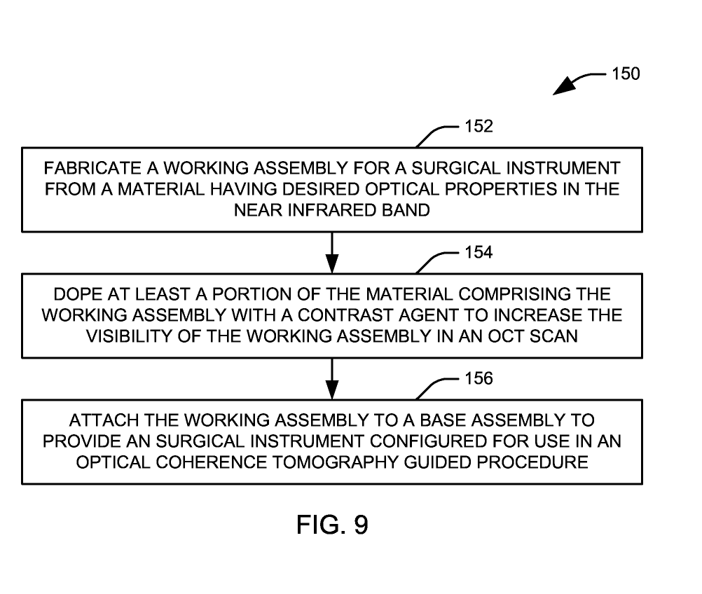

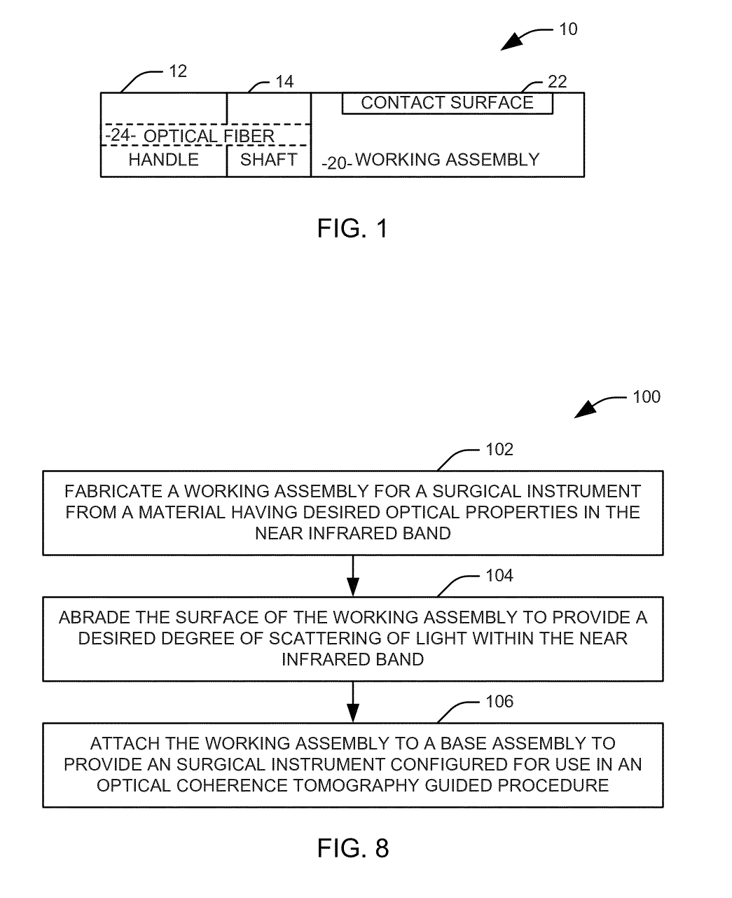

FIG. 1 illustrates an abstract, functional block diagram of a surgical instrument 10 for use in optical coherence tomography (OCT) assisted procedures in accordance with an aspect of the present invention. The instrument 10 includes a handle 12 and a shaft 14 connecting the handle to a working assembly 20. The handle 12 is designed to be securely and comfortably grasped by a surgeon's hand. The handle 12 and shaft 14 can be made of any suitable material sufficient durable for a surgical instrument. The working assembly 20 has a contact surface 22 intended to contact the tissue during the surgical procedure. Exemplary instruments intraocular ophthalmic forceps, an ophthalmic pic, horizontal/vertical scissors, keratome blades, vitrectors, lamellar corneal needles, trephines, and subretinal needles, although it will be appreciated that other devices are envisioned in accordance with an aspect of the present invention.

In accordance with an aspect of the present invention, the working assembly 20 can be designed such that it does not significantly interfere with the transmission of infrared light between the eye tissue and the OCT sensor. Specifically, the working assembly 20 can be formed from a material having appropriate optical and mechanical properties. In practice, the working assembly is formed from materials that are optically clear (e.g., translucent or transparent) at a wavelength of interest and have a physical composition (e.g., tensile strength and rigidity) suitable to the durability and precision need of surgical microinstruments. Exemplary materials include but are not limited to polyvinyl chloride, glycol modified poly(ethylene terephthalate) (PET-G), poly(methyl methacrylate) (PMMA), and a polyphenylsulfone, such as that sold under the brand name RADEL™.

In one implementation, the material of the working assembly is selected to have an index of refraction, for the wavelength of light associated with the OCT scanner (e.g., between 750 nm and 1400 nanometers), within a range close to the index of refraction of the eye tissue media (e.g., aqueous, vitreous). This minimizes both reflection of the light from the instrument and distortion (e.g., due to refraction) of the light as it passes through the instrument. In this implementation, the index of refraction of the material is selected to be between 1.3 and 1.55. The material is also selected to have a scattering coefficient within a desired range, such that the instrument is visible within the image, but does not obscure the tissue underneath the instrument. In this implementation, a scattering coefficient between 2 mm⁻¹ and 5 mm⁻¹ is desirable. A material can also be selected according to an associated attenuation coefficient to ensure that sufficient light passes through to allow for imaging of the underlying tissue. Since attenuation is a function of the thickness of the material, the attenuation coefficient of the material used may vary with the specific instrument or the design of the instrument. For example, polyvinyl chloride has excellent transmittance of infrared light, and an index of refraction in the near infrared band (e.g., 0.75-1.4 microns) around 1.5. It has a tensile modulus of around 2400 MPa, and a scattering coefficient of around 2.7 mm⁻¹ in that band.

The inventor has determined that several materials with otherwise desirable properties provide insufficient diffuse reflectivity for a desired clarity of visualization of the instrument during an OCT scan. For example, certain transparent plastics have an amorphous microscopic structure and do not provide a high degree of diffuse scattering in the infrared band. In accordance with another aspect of the present invention, a surface of the working assembly can be abraded or otherwise altered in texture to provide a desired degree of scattering, such that the instrument is visible in the OCT scan without shadowing the underlying tissue. In one implementation, this shading is limited to the contact surface 20 to provide maximum clarity of the tissue within the scan, but it will be appreciated that, in many applications, it will be desirable to provide surface texturing to the entirety of the surface of the working assembly 14 to allow for superior visibility of the instrument.

In yet another implementation, a contrast agent can be introduced to the material via doping to improve the visibility of the working assembly 14 in the OCT scan. In such a case, an external light source, having a wavelength different than that of the OCT scanner, may be used to facilitate identification of the contrast agent, as is described in more detail below. Various types of contrast agents can include microparticles, nanoparticles, substances compatible with pump-probe techniques, spectroscopic contrast agents, and nonlinear or polarization-sensitive agents.

One implementation of an intraoperative instrument using semi-transparent material doped with optical contrast can use nanoparticles are tuned to have a plasmon resonance that overlap with a frequency of a pump laser, resulting in strong optical absorption and photothermal heating. For example, the pump light source can also be sent through optics associated with a surgical scope, be aligned to be collinear with the OCT imaging light, and/or integrated into the instrument itself. The local heating causes changes in the index of refraction that can be detected using OCT as a phase-shift, which can be quantified using photothermal OCT or Doppler OCT methods. Heterodyne or lock-in detection can be used to filter out extraneous background scatters to achieve detection sensitivities of fourteen parts per million, which allows the use of low concentration nanoparticle doped materials, thus reducing any potential of increased scattering. Additionally, nanoparticles of different shapes can be used to provide different absorption and scattering properties, including nanoshells, nanospheres, nanorods, and nanocages.

In addition to plasmon resonance, nanoparticle size, shape, and material can be specified to achieve varying absorption and heating characteristics and optical properties, such as the use of nanorods to achieve low scattering in OCT while retaining high photothermal efficiency, and the use of carbon instead of conventional metallic nanoparticles for multimodal imaging. Quantum dots can also be used to achieve photothermal expansion and can be implemented similar to nanoparticles. In addition to optical excitation, ferromagnetic particles can be used as a means of achieving optical contrast. Here, the optical fiber and pump laser can be replaced with an electromagnet, which will provide oscillating magnetic fields at a heterodyne frequency. Similar to the photothermal implementation, these oscillations result in local perturbations of the ferromagnetic particles, which can be detected using phase-sensitive OCT methods.

In a specific implementation, the transparent material at the tip of the instrument is doped with gold nanorods tuned with a plasmon resonance outside of the OCT imaging bandwidth. The instrument can include an optical fiber 24 along its handle 12 and shaft 14 that relays a pump laser, matched to the resonance wavelength of the nanorods, to the tip where it scatters in the semi-transparent substrate, is absorbed by the nanoparticles, results in temperature changes around the particle, and leads to variations in the local index of refraction. The pump laser is frequency modulated to allow for heterodyne or lock-in detection of minute photothermal changes in materials with low nanoparticle concentration.

In a second implementation, pump-probe imaging methods can be used to achieve optical contrast. In this implementation, molecules with complex electronic and vibrational states can be used as the dopant. Accordingly, each of the OCT probe laser and an external light source, like the pump laser used with the nanoparticles, are implemented as pulsed laser sources and delayed with respect to each other to match the ground state recovery time of the contrast agent. Changes in the attenuation of the OCT probe beam are used to identify concentrations of the contrast agent driven to the excited state by the pump beam. Like the pump laser used with the nanoparticles, the pump beam is frequency modulated to improve detection sensitivity.

Wavelength-dependent attenuation of the OCT imaging beam can also be used to identify spectroscopic contrast agents with preferential absorption within the imaging bandwidth. To this end, the contrast agent is selected such that the OCT bandwidth and absorption spectrum of the dopant overlap, and no external light source is used. By looking at the spectral shape of the detected OCT signal as a function of depth, preferential absorption bands can be identified using wavelength demultiplexing algorithms. The use of preferential spectroscopic absorption, such as fluorescent compounds, nanoparticles, or quantum dots, has advantages over photothermal methods because no heterodyned excitation/detection is required and measurements can be made on individual interferograms without the need for repeated scans. The use of a semi-transparent substrate is particularly advantageous for spectroscopic contrast agents because the surface reflections of the material can be used to make relative absorption measurements of the doped regions. This avoids many artifacts common to spectroscopic imaging due to wavelength-dependent absorption of blood and tissue common in in vivo applications.

Finally, contrast agents that exhibit strong optical nonlinearity or polarization can also be used as material dopants for OCT friendly instrumentations. Some examples include non-centrosymmetric compounds that exhibit a second harmonic signal detectable by OCT and materials with detectable birefringence, depolarization, or phase retardance, which may be measured using polarization-sensitive OCT. This class of contrast agents is particularly attractive because these material properties originate from microscopic alignments in material fiber orientation or molecular orientation and, therefore, would not have any effect on the transmissivity of the material.

A significant advantage of using substrate-doped contrast agents for OCT instruments is it allows for in vivo application of exogenous contrast agents without the dangers of toxicity and clearance. Additionally, since there will likely not be any dynamic changes in the concentration of any dopants, materials may be identified using relative measurements, which eliminates the need for cumbersome calibration measurements common to spectroscopic and polarization measurements. Finally, since all of the described contrast agents can be tailored to specific operating regimes, the use of doped materials does not preclude the concurrent use of additional contrast agents, such as fluorescent dyes or inks, common to surgical procedures.

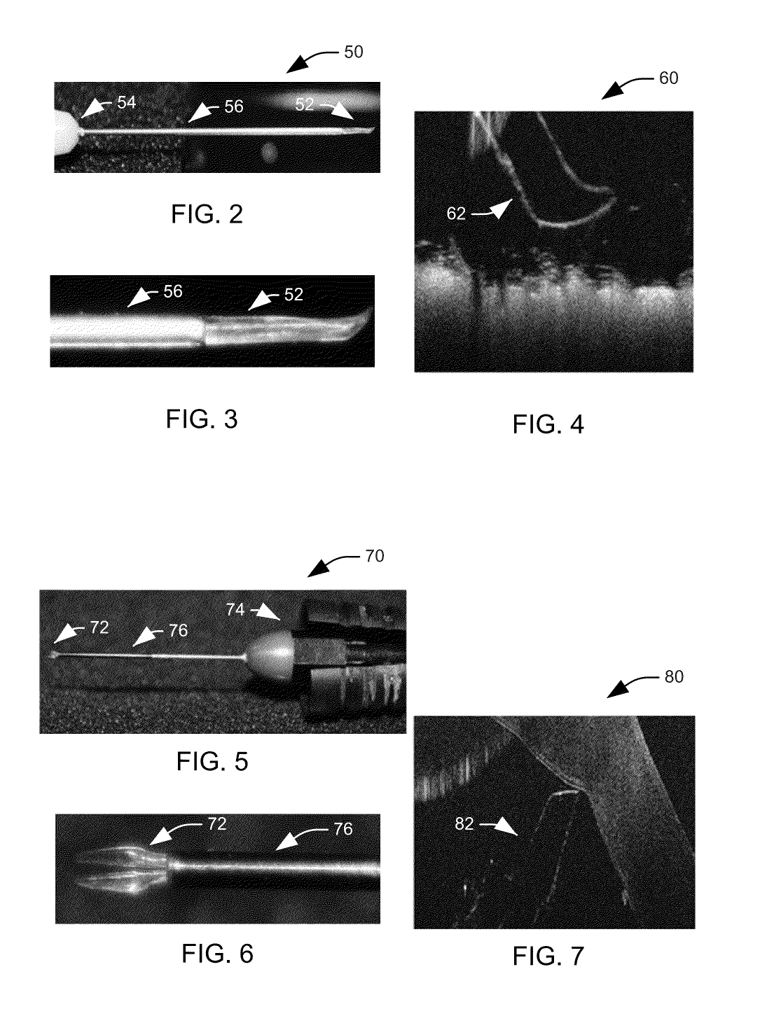

FIG. 2 illustrates a first example of a surgical instrument 50, specifically an ophthalmic pic, in accordance with an aspect of the present invention. FIG. 3 provides a close-up view of a working assembly 52 associated with the instrument 50. The instrument 50 has a handle 54 configured to be easily held by a user and a shaft 56 connecting the working assembly 52 to the handle. The working assembly 52 formed from a clear plastic and can, optionally, have surfacing applied to increase the diffuse reflection provided by the clear plastic. FIG. 4 illustrates an OCT scan 60 of a region of eye tissue with the ophthalmic pic 50 of FIGS. 2 and 3 interposed between the OCT scanner and the tissue. A shadow 62 of the instrument is visible in the OCT scan 60, but it will be noted that the tissue under the instrument remains substantially visible.

FIG. 5 illustrates a second example of a surgical instrument 70, specifically ophthalmic forceps, in accordance with an aspect of the present invention. FIG. 6 provides a close-up view of a working assembly 72 associated with the instrument 70. The instrument 70 has a handle 74 configured to be easily held by a user and a shaft 76 connecting the working assembly 72 to the handle. The working assembly 72 formed from semi-transparent plastic and can, optionally, have surfacing applied to increase the diffuse reflection provided by the semi-transparent plastic. FIG. 7 illustrates an OCT scan 80 of a region of eye tissue with the ophthalmic forceps 70 of FIGS. 5 and 6 interposed between the OCT scanner and the tissue. Again, a shadow 82 of the instrument is visible in the OCT scan 80, but it will be noted that the tissue under the instrument remains substantially visible.

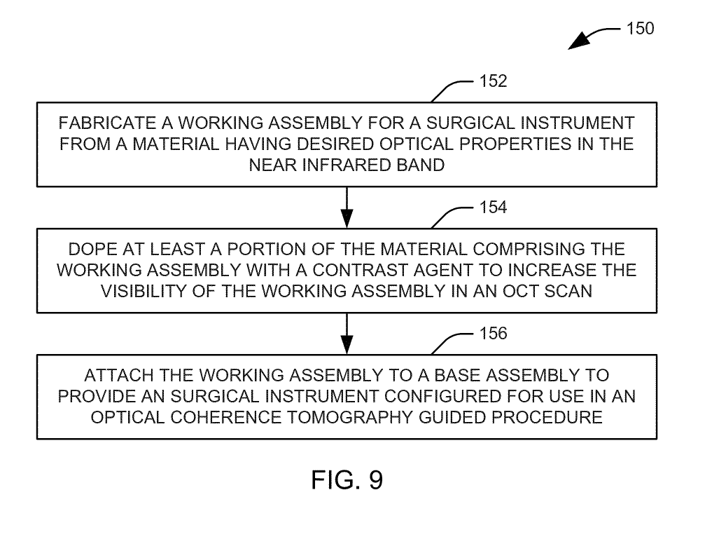

In view of the foregoing structural and functional features described above, methodologies in accordance with various aspects of the present invention will be better appreciated with reference to FIGS. 8 and 9. While, for purposes of simplicity of explanation, the methods of FIGS. 8 and 9 are shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect the present invention.

FIG. 8 illustrates a method 100 for constructing an instrument for use in OCT assisted surgical procedures in accordance with an aspect of the present invention. At 102, a working assembly for the instrument is fabricated from a material having desired optical properties in the near infrared band. For example, these properties can include an index of refraction near that of the tissue to be operated upon and a minimal absorption at the wavelength associated with the OCT scanner. At 104, the surface of the working assembly is abraded to increase the scattering of the near infrared light from the working assembly. Depending on the implementation and configuration of the working assembly, the abrasion can be substantially random or performed in a specific pattern (e.g., a grating) to provide a desired degree of reflection. At 106, the working assembly is attached to a base assembly (e.g., a handle and/or shaft) to provide the surgical instrument.

FIG. 9 illustrates a method 150 for constructing an instrument for use in OCT assisted surgical procedures in accordance with an aspect of the present invention. At 152, a working assembly for the instrument is fabricated from a transparent or semi-transparent material. In one implementation, the semi-transparent material is selected for desirable mechanical properties but may have a scattering coefficient too low to be clearly visible in an OCT scan. At 154, the material of the working assembly is doped with a contrast agent to increase the visibility of the working assembly in an OCT scan. The contrast agent is selected to enhance the optical contrast of semi-transparent material under tunable conditions, such as over a particular wavelength range or for specific external excitation. At 156, the working assembly is attached to a base assembly (e.g., a handle and/or shaft) to provide the surgical instrument.

From the above description of the invention, those skilled in the art will perceive improvements, changes, and modifications. Such improvements, changes, and modifications within the skill of the art are intended to be covered by the appended claims.

Having described the invention, we claim:

1. An assembly for use as a surgical instrument in a surgical procedure guided by an optical coherence tomography (OCT) system having a light source with an associated wavelength within the near infrared range, the assembly comprising:
a working assembly formed from a semi-transparent plastic selected to have an index of refraction between 1.3 and 1.55 at the associated frequency and a scattering coefficient between 2 mm⁻¹ and 5 mm⁻¹ at the associated frequency; and
a handle attached to the working assembly.

2. The assembly of claim 1, wherein the semi-transparent plastic is polyvinyl chloride.

3. The assembly of claim 1, wherein the semi-transparent plastic is doped with a contrast agent to increase a visibility of the working assembly in an OCT scan.

4. An assembly for use as a surgical instrument in a surgical procedure guided by an optical coherence tomography (OCT) system having a light source with an associated frequency within the near infrared range, the assembly comprising:
a working assembly formed from a semi-transparent plastic doped with a contrast agent selected to improve the visibility of the working assembly in an OCT scan; and
a handle attached to the working assembly.

5. The assembly of claim 4, the contrast agent comprising nanoparticles tuned to have a plasmon resonance that does not overlap with the associated frequency of the light source but does overlap with a frequency of a pump laser associated with the assembly.

6. The assembly of claim 5, wherein the contrast agent comprises a metallic nanoparticle.

7. The assembly of claim 6, wherein the contrast agent comprises a gold nanoparticle.

8. The assembly of claim 5, wherein the contrast agent comprises a carbon nanoparticle.

9. The assembly of claim 5, further comprising an optical fiber passing through the handle to relay an output of the pump laser to the working assembly.

10. The assembly of claim 4, wherein the contrast agent comprises quantum dots.

11. The assembly of claim 4, wherein the contrast agent comprises ferromagnetic particles.

12. The assembly of claim 4, wherein the contrast agent comprises a spectroscopic contrast agent having an absorption spectrum overlapping the associated frequency of the light source.

13. The assembly of claim 12, wherein the contrast agent comprises a fluorescent compound.

14. The assembly of claim 4, wherein the contrast agent is selected to have an significant nonlinear optical response at the associated frequency of the light source.

15. The assembly of claim 14, wherein the contrast agent comprises a non-centrosymmetric compound that exhibits a higher order harmonic signal detectable by the OCT system in response to light at the associated frequency of the light source.

16. The assembly of claim 4, wherein the contrast agent comprises a material exhibiting one of birefringence, depolarization, and phase retardance of light at the associated frequency of the light source.

17. The assembly of claim 4, wherein the light source is a first light source and the assembly further comprises an optical fiber passing through the handle to relay an output of a second light source to the working assembly.

18. An assembly for use as a surgical instrument in a surgical procedure guided by an optical coherence tomography (OCT) system having a light source with an associated frequency within the near infrared range, the assembly comprising:
a working assembly formed from a semi-transparent plastic comprising one of glycol modified poly(ethylene terephthalate, polyvinyl chloride, poly(methyl methacrylate), and polyphenylsulfone; and
a handle attached to the working assembly.

19. The assembly of claim 18, wherein the semi-transparent plastic is doped with a contrast agent selected to improve the visibility of the working assembly in an OCT scan.

20. The assembly of claim 18, wherein the light source is a first light source and the assembly further comprises an optical fiber passing through the handle to relay an output of a second light source to the working assembly.
